(19) United States
(12) Patent Application Publication
(10) Pub. No.: US 2007/0276339 A1
(43) Pub. Date: Nov. 29, 2007

(54) METHOD AND APPARATUS FOR PROVIDING HAND OPERATED PORTABLE SUCTION

(76) Inventor: Jason Pickett, (US)

Correspondence Address: NIXON & VANDERHYE, PC, 901 NORTH GLEBE ROAD, 11TH FLOOR, ARLINGTON, VA, 22203, US

(21) Appl. No.: 11/752,690

(22) Filed: May 23, 2007

Related U.S. Application Data
(60) Provisional application No. 60/747,956, filed on May 23, 2006.

(51) Int. Cl. A61M 5/00

(52) U.S. Cl. 604/187

(57) ABSTRACT

A Hand Operated Portable Suction (HOPS) unit is placed in either hand for utilization. The thumb is placed partially through a center ring and other digits are placed through flanges. The nose is grasped with the other hand and pulled to extend it, then directed into a usable shape for operation as the operator prefers. The aspirating end is directed by the user into a pool of liquid material to be suctioned or against a solid piece of material that is to be removed using suction. The thumb is extended, withdrawing the plunger from the barrel. This creates a cavity in the barrel into which liquid and semi-solid material is drawn. The plunger stopper maintains the seal against the interior surface of the barrel in order to ensure that air and liquid filling the cavity is drawn in through the nose. The nose is withdrawn from the area being suctioned and pointed in a safe direction. The thumb is then flexed, pushing the plunger into the barrel and expelling liquid and semi-solid contents of the barrel cavity through the nose.

### CROSS-REFERENCES TO RELATED APPLICATION

This application claims the benefit of priority from provisional application No. 60/747,956 filed May 23, 2006, incorporated herein by reference.

### FIELD

The technology herein relates to medical devices, and more particularly to handheld portable suction systems. Still more particularly, the technology herein relates to techniques, systems, methods of use and methods of manufacture relating to hand-operated portable suction syringes.

### BACKGROUND AND SUMMARY

Emergency medical care providers including but not limited to physicians, nurses, paramedics, and emergency medical technicians are often required to quickly remove vomit, secretions, foreign material, and blood from the airways of critically ill patients. This is typically accomplished through introduction of a suction catheter or wand into the airway. The suction catheter or wand sucks the material from the airway and removes it to a closed container.

Suction is readily available in hospital facilities of most industrialized countries. For example, many or most modern hospital rooms include a wall-mounted suction fitting to which a doctor, nurse or medical technician can attach suction devices such as a catheter. In the field environment, wall-mounted powered suction is most often unavailable, and emergency medical providers must rely on portable suction units for the task.

Portable suction units come in two general classes: battery powered units and non-powered or hand-operated units. Currently available portable suction devices have significant limitations. Battery powered units are expensive (up to $1000 per unit) and require regular charging, maintenance, and attention. The rechargeable batteries which power these units are prone to power loss when unattended, and when drained, leave the emergency provider with no means of suction. Battery operated units are also bulky and heavy, limitations which may discourage emergency providers from carrying them to the scene of an illness or injury. Their large size make them impractical for use in a tactical environment such as with police tactical teams or military special operations units.

Hand-operated or other non-powered units use hand power from the operator to generate suction. These units have the advantage of being smaller in size as compared to the battery powered units, and may be small enough to carry with other supplies in large trauma bags. They also do not depend on batteries or other electrical power sources. Hand-operated units are frequently carried by emergency providers as a back-up to use if powered units should fail. However, currently available hand-operated suction units have certain limitations. For example, handheld units may have limited capacity for volume of aspirate and limited suction force. Some providers complain that handheld units do not provide adequate suction for use. The most widely used units are generally too large for practical use in the tactical environment, where space may be limited to a vest or small pouch. Handheld units, while less expensive than battery powered units, can still cost $100 each.

Military medics and tactical emergency medical providers require a reliable means of providing suction that is compact, lightweight, simple to operate, inexpensive, and can withstand the extreme environments and rugged use of modern tactical operations.

The technology herein is designed to fill this need. An exemplary illustrative non-limiting implementation of a hand-operated tactical suction unit is very compact (e.g., less than 6 inches long and 1.5 inches wide). A central plunger inserts into a clear plastic barrel permitting contents to be visually examined. The plunger has a stopper which provides contact with the inner surface of the barrel and forms an airtight seal. The control end of the plunger has a ring for the operator's digit (e.g., thumb) so the plunger can be manipulated in both directions using one hand. The barrel has rings or flanges affixed opposite each other at the control end, for insertion of other digits (e.g., the index and middle or index and ring fingers) to facilitate one-handed operation. A flexible collapsible hollow corrugated trunk, hose or catheter portion is attached at the operating end of the barrel. The corrugated trunk may be manipulated and directed at the operator's discretion and will maintain its shape once configured. The trunk has an orifice at the operating end. Liquid may be aspirated through the orifice under vacuum pressure and/or solid material may be grasped through vacuum pressure. The entire apparatus, with the exception of the stopper, may be constructed of lightweight translucent or transparent plastic to facilitate visualization of contents and resist damage in hostile environments.

### BRIEF DESCRIPTION OF THE DRAWINGS

These and other features and advantages will be better and more completely understood by referring to the following detailed description of exemplary non-limiting illustrative implementations in conjunction with the drawings of which:

FIG. 1A is a cross-sectional side view of an exemplary illustrative non-limiting implementation of a hand operated portable suction device;

FIG. 1B is a plan view of the FIG. 1A implementation compacted for storage;

FIG. 1C shows a front view of the FIG. 1A implementation;

FIG. 1D is another plan view of the FIG. 1A implementation with the nose providing an extendable accordion-like elongated tubular structure;

FIG. 1E is a further plan view showing the plunger retracting in use to apply suction;

FIG. 1F illustrates a hand operating the FIG. 1A device;

FIG. 2A is a cross-sectional side view of an exemplary illustrative non-limiting implementation of a hand operated portable suction device with a curved snout;

FIG. 2B is a plan view of the FIG. 2A implementation;

FIG. 2C shows a front view of the FIG. 2A implementation;

FIG. 2D is another plan view of the FIG. 2A implementation with the nose providing an extendable accordion-like elongated structure; and

FIG. 3 shows an exemplary illustrative non-limiting further implementation using finger rings instead of disks.

### DETAILED DESCRIPTION

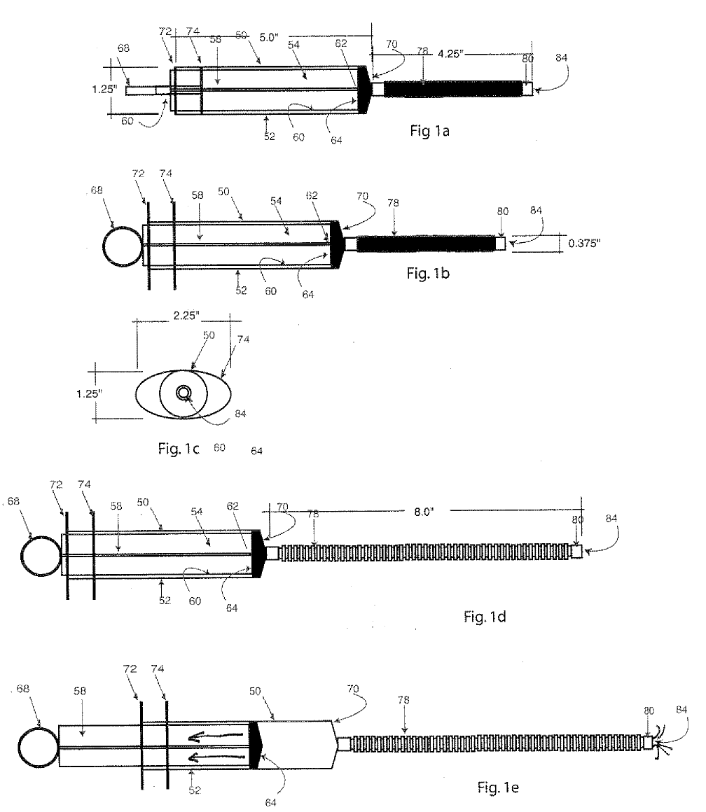

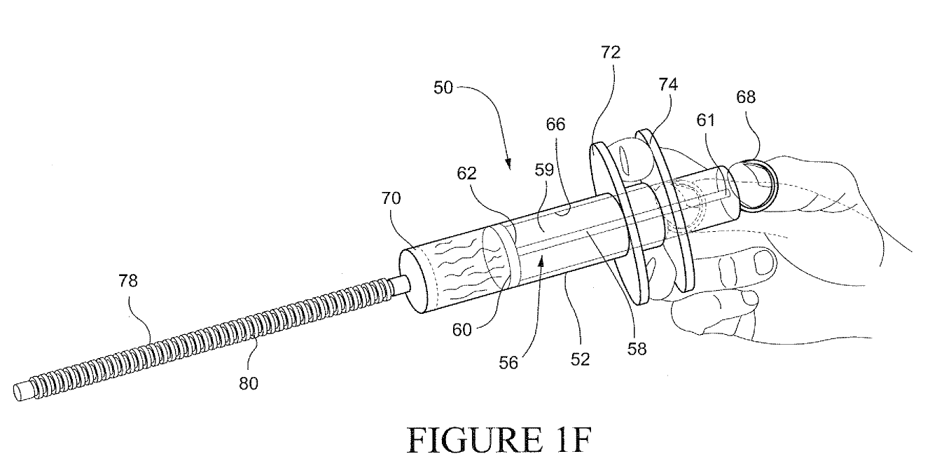

FIG. 1A shows an exemplary illustrative non-limiting implementation of a hand-operated tactical suction device 50. Device 50 may comprise a cylindrical barrel housing 52 defining a hollow chamber 54 therein. Barrel housing 52 may comprise hard, preferably clear or see-through plastic such as polycarbonate or any other suitable material. Barrel housing 52 may for example resemble the barrel of a conventional large bore (e.g., 60 cc) syringe but with a cross-sectional diameter of 38 mm.

A plunger assembly 56 is disposed within the chamber 54. The plunger assembly 56 includes a shaft 58 having a first end 60 and a second end 62. A stopper 64 is disposed at the shaft second end 62. Stopper 64 conforms closely to the housing inner cylindrical wall 66 and acts as a piston to seal against the wall and provide a vacuum pumping action as described below.

The stopper 64 conforms with seals against the housing inner cylindrical wall 66. Stopper 64 may be comprised of latex rubber, deformable soft plastic or any other suitable sealing material. Stopper 64 may be pancake-shaped or dimensioned and structured in any other convenient way as known to those skilled in the art.

As shown in FIG. 1B, a handle 68 is disposed on shaft first end 60. Handle 68 may comprise a ring (or other grasping structure). Ring 68 may be dimensioned to accept the thumb or finger of an average person (e.g., 1 inch in diameter). Inserting a finger or thumb into ring 68 (see FIG. 1F) allows the user to reposition the plunger stopper 64 to any desired position within the barrel housing 52. Pulling ring 68 outwardly away from housing 52 reduces the air pressure within a “nose” portion 70 of the chamber 54 within housing 52, causing a partial vacuum to be formed. The amount of vacuum depends for example the cross-sectional diameter of barrel housing 52. Such vacuum is used to create suction at a desired point of aspiration as will be explained.

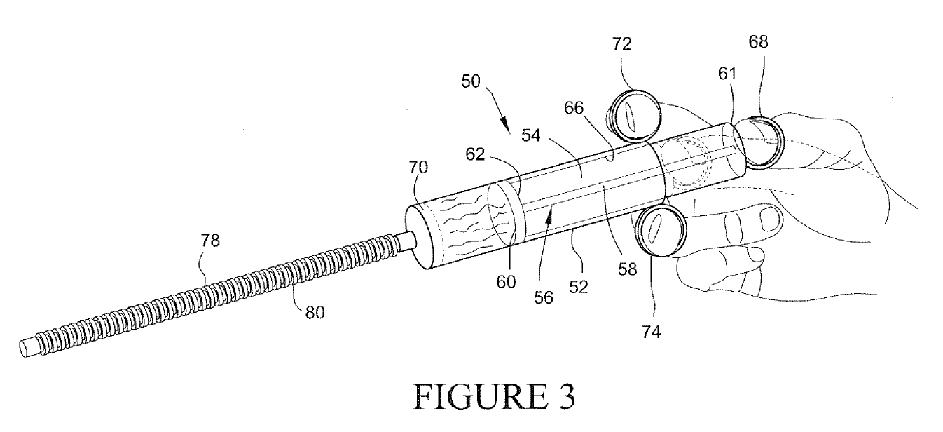

One or more finger retaining structures such as flanges 72, 74 disposed on an end 76 of housing 52 accept and retain other fingers (e.g., the middle or index finger and the ring finger) to facilitate one-handed operation. While flanges are used in this particular exemplary illustrative non-limiting implementation, rings (see FIG. 3) or other structures could be used instead. These other fingers inserted between flanges 72, 74 are used to hold, guide, direct and retain the device 50 and also provide additional leverage for relative motion between the thumb and other digits and thus between ring 68 and flanges 72, 24. This relative motion and additional leverage allows the user to more easily apply force to plunger 56. One-handed operation can be useful in many tactical and other situations.

In one exemplary illustrative non-limiting implementation, an elongated catheter or trunk portion 78 is coupled to the barrel housing nose 70. Elongated catheter or trunk portion 78 may comprise a hollow tube 80 having an extendable, bendable tubular portion 82. The catheter or trunk portion 78 thus defines a elongated passage 84 therethrough (see FIG. 1C) in fluid communication with chamber 54 and thus with vacuum developed within the chamber as plunger 56 is retracted.

To save space, catheter or trunk portion 78 is initially retracted and/or retractable in the exemplary illustrative non-limiting implementation. FIG. 1A shows the catheter extendable portion 82 in a retracted or folded-up (i.e., initial) position. FIG. 1D shows the extendable catheter portion 82 in an extended position. The extendable portion 82 may extend to on the order of twice as long (or more) in the extended position as compared to the retracted position. For example, the extendable portion 82 may have an unextended length of 4.25 inches as compared to an extended length of 8.0 inches.

In one exemplary illustrative non-limiting implementation, extendable portion 82 may comprise an accordion-like or corrugated tubular structure comprised of flexible plastic or other material. Folds within extendable portion 82 may be unfolded to extend the portion to nearly twice its retracted length. In other exemplary illustrative non-limiting arrangements, extendable portion 82 may be implemented using coaxial tubes (e.g., one sliding inside the other), stretchable tubular material with memory, or any other hollow tubular material that will retain an elongated extended shape when pulled.

In use, a medical technician or other user may unwrap suction device 50 from a sterile shrink wrap or other package (not shown), grasp the catheter portion 78 and pull it to extend the extendable portion 82 to its extended position as shown in FIG. 1D. The catheter portion 78 is non-elastically deformable in the illustrative exemplary non-limiting implementation, i.e., it can be deformed such as by bending and it will hold its shape upon release. If desired, the medical technician can bend the extended catheter portion 78 (which may be termed a “trunk” portion because in this implementation it resembles an elephant's trunk) to any desired arc, curve or other configuration including for example “S curves”, spirals, angles, bends or the like and the extendable portion will retain the shape it is bent or twisted into. The medical technician may then insert his or her fingers and thumb into the ring 68, and between flanges 72, 74 as shown in FIG. 1F, and position the far end 84 of catheter 78 into an airway, wound or other area that needs to be suctioned or aspirated. The medical technician will then move his or her thumb to retract plunger 56 within the barrel to create negative pressure by extraction of plunger (See FIG. 1E). Generated vacuum is applied to the trunk portion 78. As the plunger 56 is displaced through a volume of chamber 54, an equal volume of gas or liquid may be sucked into and through the catheter 78 into chamber 54 (see FIG. 1E view with the plunger partially extracted and nose extended for operation) Alternatively, the suction can be used to pick up solid objects.

If necessary, the medical technician can move catheter 78 over a waste receptacle, reverse the movement direction of plunger 56 to expel the previously suctioned material into the receptacle, and repeat the above operations to suction more material from the airway, wound or other area.

In more detail, a Hand Operated Portable Suction (HOPS) is placed in either hand for utilization. The thumb is placed partially through the center ring 68, The index finger and middle or ring finger are placed between flanges 72, 74 in the figure. See FIG. 1F. While flanges are used in this particular exemplary illustrative non-limiting implementation, rings or other structures could be used instead. The nose is grasped with the other hand and pulled to extend it, then directed into a usable shape for operation as the operator prefers. The aspirating end is directed by the user into a pool of liquid material to be suctioned or against a solid piece of material that is to be removed using suction. The thumb is extended, withdrawing the plunger from the barrel. This creates a cavity in the barrel into which liquid and semi-solid material is drawn. The plunger stopper maintains the seal against the interior surface of the barrel in order to ensure that air and liquid filling the cavity is drawn in through the nose. The nose is withdrawn from the area being suctioned and pointed in a safe direction. The thumb is then flexed, pushing the plunger into the barrel and expelling liquid and semi-solid contents of the barrel cavity through the nose.

In one exemplary illustrative non-limiting implementation, dimensions of rings 68, and flanges 72, 74 can be for example 1.25″ diameter ring for finger and 1.25″ diameter ring for thumb. Between these flanges the index and middle fingers may be placed, providing stabilization for operation of the plunger by the thumb. The aspiration nose may comprise corrugated accordion-like plastic that is extendable and positionable. The plunger shaft may comprise rigid plastic made of two intersecting perpendicular strips. The plunger stopper may comprise flexible plastic sized to seal against the inside of the barrel.

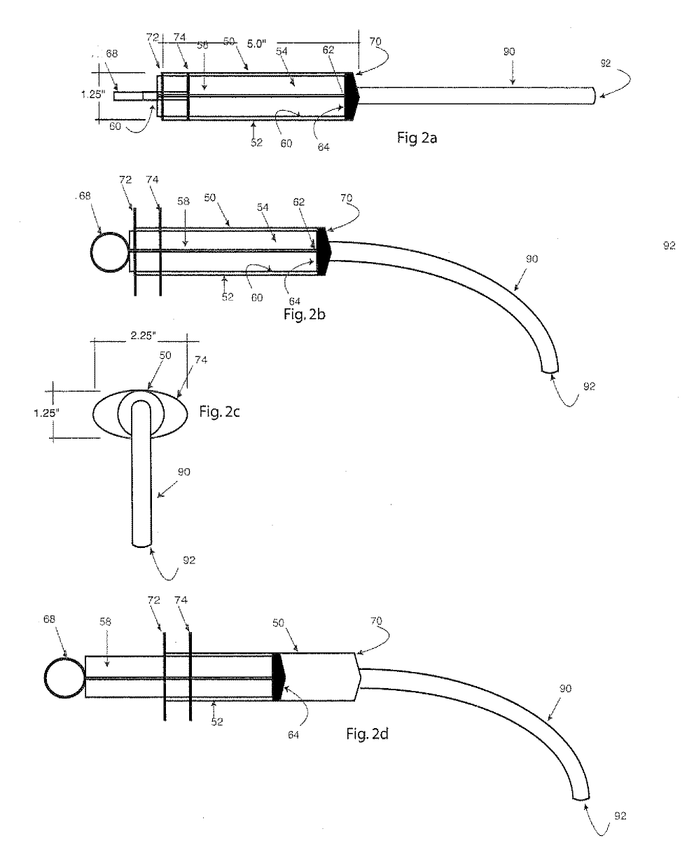

FIGS. 2A-2D show an exemplary illustrative variation of the FIG. 1A portable suction device including a curved, non-extendable snout (90) for aspiration through the hollow end (92).

It is also possible to use different configurations for the plunger. Many syringes, rather than using a barrel shaped plunger, use two intersecting planes of plastic shaped in an “x” for stability and reduced weight. Such a design feature may be included to allow flexibility in manufacture.

While the technology herein has been described in connection with exemplary illustrative non-limiting implementations, the invention is not to be limited by the disclosure. The invention is intended to be defined by the claims and to cover all corresponding and equivalent arrangements whether or not specifically disclosed herein.

We claim:

1. A one-hand-operable suction device comprising:
an elongated barrel portion having inner walls defining a cavity therein;
a plunger slidably disposed within said barrel portion cavity, said plunger sealably contacting said barrel portion inner walls so that sliding said plunger axially with respect to said elongated barrel portion develops at least a partial vacuum within said cavity; and
an at least partially flexible elongated tube coupled to said partial vacuum within said cavity, said elongated tube applying suction to an end thereof in response to said developed partial vacuum.

2. The device of claim 1 wherein said elongated tube is retractable.

3. The device of claim 1 wherein said elongated tube is corrugated.

4. The device of claim 1 wherein said elongated tube is expandable to on the order of at least twice its nominal length by pulling.

5. The device of claim 1 wherein said elongated tube comprises hollow, accordion-shaped flexible plastic.

6. The device of claim 1 further comprising a thumb ring mechanically coupled to said plunger, said thumb ring accepting and retaining a thumb.

7. The device of claim 1 wherein said elongated tube is non-elastically deformable by hand.

8. The device of claim 1 further including graspable structures disposed on said plunger for allowing a user to manually grasp and hand operate said plunger.

9. The device of claim 8 wherein said graspable structures comprise flanges.

10. The device of claim 8 wherein said graspable structures comprise rings.

11. The device of claim 1 wherein said tube is dimensioned for insertion into a subject's windpipe.

12. A method of applying suction to a wound comprising:
grasping a portable device with one hand, including placing the thumb within a ring and placing first and second fingers within a finger retaining arrangement;
placing a sterile tube projecting from the device onto the wound;
moving the thumb rearwards of the device to thereby withdraw a piston from the device, said piston withdrawal causing the device to partially evacuate a chamber therein and apply a negative pressure to the tube; and
drawing material from the wound into the tube with said negative pressure.
